(19) United States
(12) Patent Application Publication
(10) Pub. No.: US 2007/0026481 A1
(43) Pub. Date: Feb. 1, 2007

(54) Toxic mold screening kit & method for the colorimetric determination of the presence or absence of the toxic mold stachybotrys chartarum and other toxic environmental molds

(76) Inventors: Sean Michael Reilly, E. Stroudsburg, PA (US); Paul Thomas La Rocca, Sparta, NJ (US); Mary Anne Kunz La Rocca, Sparta, NJ (US)

Correspondence Address: SEAN M. REILLY, 12 WILSON DR, SPARTA, NJ, 07871, US

(21) Appl. No.: 11/193,578

(22) Filed: Jul. 29, 2005

(51) Int. Cl. C12Q 1/04; C12M 3/00

(52) U.S. Cl. 435/34; 435/287.1

(57) ABSTRACT

A method for the presumptive detection of the presence or absence and relative dominance of the toxic mold Stachybotrys chartarum collected from an environmental surface via a swab or other sampling device. The method of growth and color development of the sample utilizes dehydrated biological growth plates, pre measured hydration ampules, a liquid spreader press and a mold growth color key chart. The development of a characteristic shade of brown-colored mold colonies, unique to the Petrifilm™ YM biological growth plate, confirms the presence of Stachybotrys chartarum. The absence of the color permits the conclusion that Stachybotrys chartarum is absent in the sample. This color development method may be used to presumptively identify other molds commonly found growing on wet, building materials.

### FIELD OF INVENTION

This invention pertains to the field of microbiological test kits and methods. Particularly it pertains to a method of self-detection of the presence or absence of a toxic environmental mold by color.

### BACKGROUND

There is a long history of methods of collection, growth and identification of environmental microorganisms. The methods of collection of samples from environmental surfaces range from swabs to tape to wipes to direct agar plate contact and the air with filters. These methods are well known to those skilled in the art.

The methods of transfer to a growth medium, incubation and enumeration and identification of environmental microbiological samples are well known to those experienced in the art.

The identification of mold by standard analytical methods of those familiar with the art includes the aspect of color development of colonies of mold growing on a variety of medias on biological growth plates.

Discussions in the literature ascribe different colors to different molds growing on a variety of biological growth plates. One such reference indicates that Stachybotrys chartarum, when grown on potato dextrose agar and cornmeal agar in standard petri dishes, produced a “dark brown/black” colony (Billips, et al). 3M Company (patent holders of Petrifilm™ brand biological growth plates) state that their plates contain dyes which will aid in differentiating molds with color, depending on how they metabolize the dyes (3M Company, St. Paul, Minn.).

They do not, however, indicate any specific color associated with any particular mold. 3M product literature mentions color with respect to blue-green or off-white colored yeast on its Petrifilm™ brand mold/yeast plates. It further states that mold colonies are usually blue in color but they may also assume their natural pigmentation, i.e. black, yellow, green, etc. It is important to note that the natural color of Stachybotrys chartarum as referred to in the literature is a black mold capable of producing potent neurotoxins. There are teachings in various patent numbers (U.S. Pat. Nos. 4,587,213; 4,565,783; 5,089,413; 5,137,812; 5,232,838; RE35,286; 5,681,712; 5,635,367 and 6,770,454) that mention the development of color as a useful aspect of a device for testing microbiological samples. None of these patents, however, ascribe or claim a specific color to be associated with a specific mold as grown on the specific dehydrated microbiological growth plates claimed in these patents.

It is also instructive to note that the general use of “wet” biological growth agars in petri dishes do not include the use of dyes that will metabolize to enhance the color of the molds or differentiate one from another. The molds on these biological growth plates are said to show their natural color. There is a large scientific body of knowledge associated with the aspect of the development of natural colors of mold on “wet” agar plates.

### SUMMARY OF THE INVENTION

This invention provides a useful device for calorimetric detection of toxin-producing environmental molds including the toxic mold Stachybotrys chartarum.

The kit of the invention is comprised of a novel configuration and compilation of microbiological tools for collecting, growing and analyzing microbiological samples from environmental surfaces. It is specifically designed for use by the general public rather than only those skilled in the art of microbiological sampling and testing.

The kit of the invention as set forth in Claim # 1 is comprised of:
1. A mold color and morphology chart of pure ATCC cultures of Stachybotrys chartarum and other individual toxin-producing molds as they present themselves growing on Petrifilm™ brand biological growth plates.
2. Petrifilm™ brand biological growth plates, available from 3M Company, St. Paul, Minn., or an equivalent substitute.
3. Collection swabs, wipes or pads of varying sizes.
4. Ampules or other measured dispensing devices of buffered USP water or broths, used for hydrating the biological growth plates.
5. A hand press for spreading and confining the hydrating liquid in a defined portion of the biological growth plates.

The utility of the present invention is based on the need for an inexpensive, simple-to-use home test kit for the detection of the toxin producing mold Stachybotrys chartarum and other molds reported as toxic and commonly found on wet or flooded building materials.

The invention of a field test kit allows non-microbiologist users to obtain useful results without the cost and delay of sending field specimens to a laboratory.

The kit's unique device is a chart which is a color reproduction of various ATCC certified mold colors as they appear after hydration and incubation on Petrifilm™ brand biological growth plates.

The color chart includes Stachybotrys chartarum and other toxic molds which are commonly found on wet building materials such as sheetrock, wood, paper, cardboard, fabrics and leather.

Where ATCC cultures of toxic molds have the same or indistinguishably similar colors, the chart will provide educational information.

Where the toxic mold has a unique color and morphological fingerprint on the Petrifilm™ brand biological growth plate, the chart aids the user in making the determination as to the probable presence or absence of a specific mold.

The chart includes a companion library of information which correlates the names of molds which typically grow preferentially on various building materials i.e., wood, sheetrock, ceiling tiles, etc. and the color that pure ATCC cultures of these molds present on Petrifilm™ brand biological growth plates or equivalent substitutes.

Since the toxic mold Stachybotrys chartarum presents such a unique color on Petrifilm™ brand biological growth plates, it can be easily quantified as to its relative proportion when compared to the other molds which may be present growing on the plate.

As various toxic molds are identified, their ATCC cultures can be grown on Petrifilm™ brand biological growth plates. Their color and morphology patterns can be added to the kits library of colors on its charts.

### BRIEF DESCRIPTION OF DRAWINGS

1. FIG. 1 shows the kit components, films (FIG. 10), ampules (FIG. 9), swabs (FIG. 11), press (FIG. 7), color chart (FIG. 5) and directions (FIG. 6).

2. In FIG. 2 a sample (FIG. 13) acquired by swab (FIG. 11) is transferred to the growth medium on the biological growth plate (FIG. 10) with the translucent top cover (FIG. 12) held aloft.

3. In FIG. 3 the specified quantity of water or broth (FIG. 9) is applied to hydrate the dehydrated biological growth medium.

4. In FIG. 4 the liquid press is applied to uniformly spread and configure the area of hydration.

5. FIG. 5 shows incubation, growth and development of color on the plate.

The preferred configuration of the kit, (FIG. 1) includes:

Petrifilm™ mold/yeast biological growth plates (3M Company, St. Paul, Minn.).

One ml pre-measured plastic, squeezable ampules of USP buffered water or broth. One ampule is required for hydrating each growth plate included in the kit. The preferred configuration of the kit includes USP buffered water.

Commercially available sanitary sampling swabs, sampling wipes or sampling pads or sampling filters which are used for the collection of the environmental samples. The wipes and filters are 37 mm diameter sterile 0.45 um filters or micro filter dust collection cloths imbedded with a static electric charge.

FIG. 4 shows a liquid-spreading hand press with an overall dimension of 3″×4″ and containing a 0.5 mm deep 1 mm wide raised rim ring on its underside and a finger grid tab on its top side. The inside diameter of the raised rim on the underside is 62 mm. The press is made of rigid translucent plastic.

A color code chart depicting the colors and typical growth patterns of the mold Stachybotrys chartarum and other toxic molds as they are viewed growing, pressed between the sheets of the Petrifilm™ brand (3M Company, St. Paul, Minn.) biological growth plate.

With reference to claims 2 through 9, the method of the invention comprises the collection of a sample of suspect mold using a sanitary swab, wipe, pad or filter according to standard methods of collection of environmental microbiological samples.

The collected sample is transferred to the biological growth plate. In the preferred method of the invention, a sample acquired by a swab is transferred to a Petrifilm™ brand (3M Company, St. Paul, Minn.) biological growth plate for mold and yeast.

The practitioner, as shown in FIG. 2, lifts the top cover sheet (FIG. 12) of the Petrifilm™ biological growth plate and rubs the swab containing the sample in an X pattern across the surface of the microbiological growth medium on the bottom sheet. The top sheet is then dropped to cover the transferred sample.

The practitioner then removes one plastic ampule of hydrating water or broth from the kit of claim 1 and clips or breaks off the tip. The preferred broth is Letheen and the preferred water is buffered USP water.

The top cover of the Petrifilm™ is lifted as shown in FIG. 3 and the full contents of the ampule are squeezed onto the center of the bottom sheet containing the transferred sample. The top cover is then dropped onto and covers the liquid.

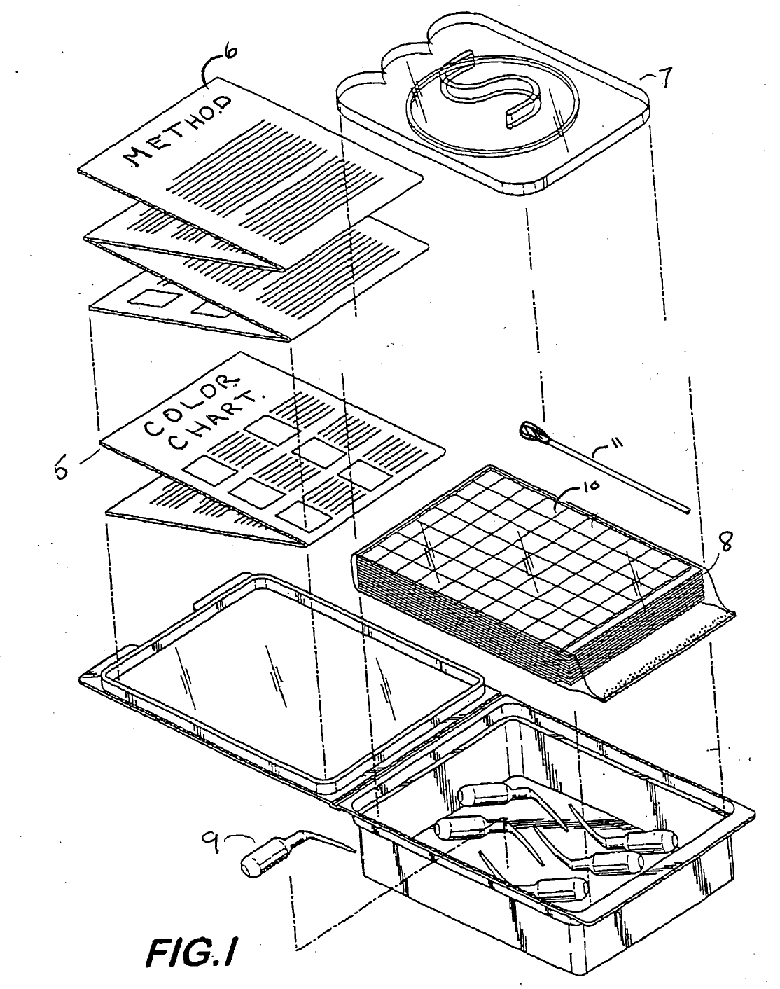

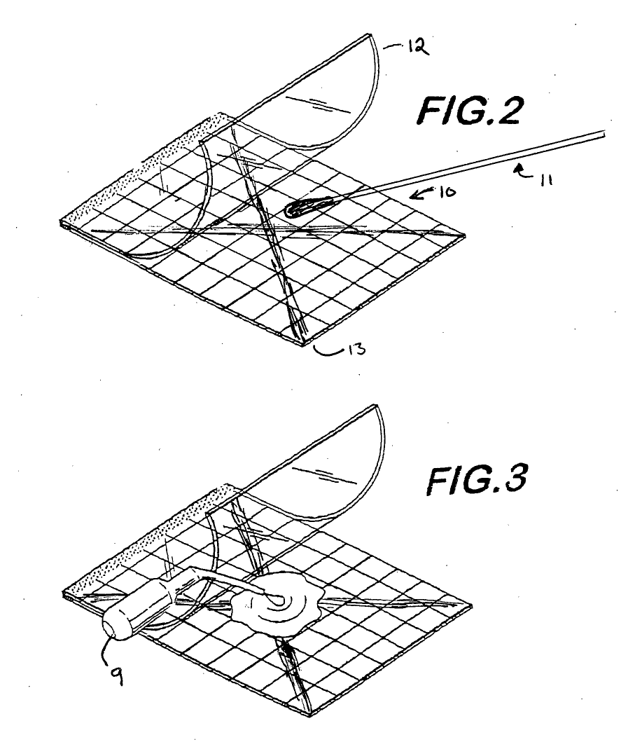

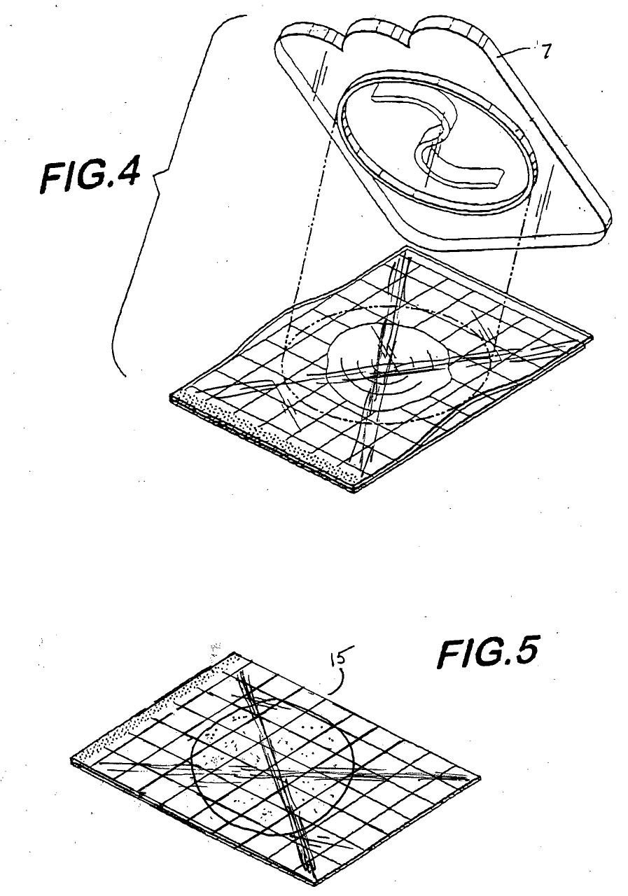

The hand press of the kit of claim 1 is centered over the droplet of hydrating liquid which is visible through the transparent top cover, FIG. 4. The translucent press is then pressed onto the top cover with a firm downward vertical pressure for about 3 seconds until the liquid spreads out between the sheets of the Petrifilm™, filling the entire confines of the circular impression the press is creating on the film. The press is removed. A circular impression of hydrated agar is confined and visible between the sheets of the Petrifilm™ biological growth plate.

The hydrated Petrifilm™ is then held in a location at room temperature for seven days in order to allow for the germination and growth of the mold sample, FIG. 5.

If the mold Stachybotrys chartarum is present, the hydrated growth area will be sprinkled with hundreds of dot-sized circular colonies ranging from olive brown to tan in color. In some instances, the mold Stachybotrys will only produce a few dot-sized colonies but will completely cover the hydrated growth area with a uniform and unique brown and tan color washover.

If other molds are present with the Stachybotrys chartarumsample, then they may display in blue, green, black, yellow, orange or red colors and are easily distinguished from the olive brown and tan colors of Stachybotrys chartarum.

After the seven days of incubation, color development of the film will be sufficient to make a determination of the presence or absence of Stachybotrys chartarum or other toxic molds.

The kit user then compares the colors and patterns on the incubated Petrifilm™ to the colors and patterns on the toxic mold color chart provided in the kit of claim 1. The toxic mold color chart includes the colors of various toxic molds grown on Petrifilm™ brand biological growth plates from certified ATCC cultures.

Based on the similarities of colors and patterns on the Petrifilm™ as compared to the mold color and pattern chart, according to the directions of the kit of Claim 1, the user makes a determination as to the number of species of molds, the absence or presence of the toxic mold Stachybotrys chartarum and relative dominance of Stachybotrys chartarum in comparison to the percentage of coverage of the hydrated circle of agar when compared against the other mold colors present and morphology confirmation.

The biological growth plate is further examined for the presence or absence of other colors and morphology, which match other toxic molds are prepared from certified ATCC cultures and grown on Petrifilm™ brand biological growth plates.

Validation

A series of swab samples of mold were acquired in the field and tested according to the method of this invention as set forth in Claims 2 through 9.

Samples of the mold were also examined under a microscope at 400× and confirmed to be Stachybotrys chartarum.

ATCC-certified cultures of Stachybotrys chartarum (ATCC #9182) were purchased and grown on Petrifilm™ brand biological growth plates.

The color and morphology (shape and pattern of the growing colony) of the Stachybotrys chartarum grown from the field samples was confirmed as the same color and morphology as the certified ATCC-certified cultures of Stachybotrys chartarum grown on Petrifilm™ mold/yeast biological growth plates.

1. (canceled)

2. A method for detecting the presence/absence of the toxic mold Stachybotrys chartarum and other toxic molds comprising:
a) Collecting a sample of suspect environmental mold from a surface using a swab, wipe or filter and
b) Transferring the sample to a biological growth plate, adding hydration, pressing closed and incubating and
c) Examining the biological growth plate on the seventh day for the presence or absence of color development indicative of the presence or absence of the mold Stachybotrys chartarum or other toxic molds in the collected sample.
d) Compare the color of the growth on the biological growth plate to the color chart of ATCC cultures of Stachybotrys chartarum and other toxic molds grown on biological growth plates.

3. A method according to claim 2, wherein the sample acquired on the swab is transferred in an X, cross (+) or an asterisk (*) pattern to the biological growth plate, which is a dry rehydratable film containing nutrients supplemented with chlortetracycline, chloramphenicol, cold H₂O-soluble gelling agent and dye sensitive to the presence of phosphatase (5-Bromo-4-Chloro-3-Indolyl Phosphate), which enhances visualization of yeast and mold growth as described in AOAC Official Method 997.002 and is available as Petrifilm™.

4. A method according to claim 2 wherein an ampule of buffered USP water or other compatible broth is added to the biological growth plate, directly on the center of the transferred sample.

5. A Method according to claim 2 wherein pressure is applied to the top cover of the biological growth plate to spread the drop of liquid between the sheets of the biological growth plate covering a defined portion of the film.

6. A method according to claim 2 wherein the hydrated biological growth plate is incubated for up to 7 days to allow for the growth and color development and morphology of molds.

7. A method according to claim 2 wherein the color development on the biological growth plate is compared against the standardized color comparison and morphology chart described in claim 10.

8. A method according to claim 2 wherein the development of the unique brown color and the morphology and dominance of mold on the biological growth plate is determined using the color chart of claim 10 and a conclusion is made as to the presence or absence of Stachybotrys and its relative dominance in the sample.

9. A method according to claim 2 wherein the color and morphology of other environmental mold samples which are grown on the Petrifilm™ brand or equivalent biological growth plate, are compared against the color chart containing the growth color presentations of other ATCC cultures of various toxic molds.

10. A method according to claim 2 wherein a color comparison chart is developed utilizing ATCC certified cultures of Stachybotrys chartarum and other toxic molds as grown on the biological growth plates described in claim 3 and photographed in color.
